(19) United States
(12) Patent Application Publication
(10) Pub. No.: US 2004/0067235 A1
(43) Pub. Date: Apr. 8, 2004

(54) Methods for the use of neurotoxin in the treatment of urologic disorders

(76) Inventor: Rajiv Doshi, Palo Alto, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/627,583

(22) Filed: Jul. 25, 2003

Related U.S. Application Data
(60) Provisional application No. 60/399,541, filed on Jul. 29, 2002.

(51) Int. Cl. A61K 39/00; A61K 39/38

(52) U.S. Cl. 424/184.1

(57) ABSTRACT

Methods and compositions for treating urological disorders are provided. In the subject methods, an effective amount of a neurotoxin is intravesically delivered into the bladder lumen of a patient in need thereof. Also provided are compositions, e.g., pharmaceutical preparations and kits, for practicing the subject methods.

### CROSS REFERENCE TO RELATED APPLICATIONS

Pursuant to 35 U.S.C. § 119 (e), this application claims priority to the filing date of the U.S. Provisional Patent Application Serial No. 60/399,541 filed Jul. 29, 2002, the disclosure of which is herein incorporated by reference.

### INTRODUCTION

1. Field of Invention

The field of this invention is urologic disorders and the treatment thereof.

2. Background of the Invention

Many urologic disorders result from dysfunction of the bladder, bladder neck, sphincter, and urethra. One such urologic disorder is urinary incontinence. Urinary incontinence is defined as the inability to stop urine leakage. There are different types of urinary incontinence. These include urge incontinence (usually associated with overactive bladder), stress incontinence, and overflow incontinence. A combination of urge and stress incontinence is known as mixed incontinence.

There are many causes of urge incontinence, several related to dysfunction of the detrusor muscle. The detrusor muscle is the smooth muscle located within the bladder wall whose contraction helps expel urine from the bladder. In the disorder known as detrusor instability, involuntary contractions of this smooth muscle leads to leakage of urine. Another cause of urge incontinence is detrusor hyperreflexia which refers to an overactive detrusor muscle caused by neurologic disorders such as multiple sclerosis, Parkinson's disease, spinal cord injury, stroke, etc. Other terms used to describe involuntary detrusor contractions which can lead to incontinence include irritable, spastic, unstable, hypertonic, uninhibited, dyssynergic, and systolic bladder.

Normally, acetylcholine (a neurotransmitter) is released from the postganglionic parasymphathetic nerves that innervate the bladder's smooth muscle. There are two types of receptors that bind acetylcholine, namely muscarinic and nicotinic receptors. Muscarinic receptors in particular are responsible for inducing excitation in smooth muscle, including smooth muscle found within the bladder wall. Medications aimed at blocking muscarinic receptors in bladder smooth muscle, including atropine and atropine-like compounds, are thereby helpful in depressing or preventing bladder contractions. That is, by blocking the muscarinic receptor, detrusor muscle activity is depressed which is helpful in treating urinary incontinence and other bladder disorders.

Various pharmacological approaches have been employed to treat urologic disorders including those involving the bladder such as urinary incontinence. Such approaches include both systemic and local delivery of various pharmacological agents. To date, none of the currently employed approaches has proven completely satisfactory, e.g., because of unacceptable side effects, because of unacceptable delivery protocols, etc.

Accordingly, there is continued interest in the development of new pharmacological approaches to treat urologic disorders. Of particular interest would be the development of local delivery protocol of an agent that provided for long-lasting treatment, such that administration would be required only infrequently. The present invention satisfies this need.

### Relevant Literature

U.S. patents of interest include: U.S. Pat. Nos. 5,674,205; 5,698,549; 6,290,961; 6,365,164. Also of interest is published U.S. application Ser. No. 2002/0025327 A1. Additional references of interest include: Cannon, TW et al., “Pharmacotherapy of the Overactive Bladder and Advances in Drug Delivery,” Clinical Obstetrics and Gynecology (2002) 45: 205-217; Dmochowski, R et al., “Advancements in Pharmacologic Management of the Overactive Bladder,” Urology (2000) 56 (Supplement 6A); Schulte-Baukloh, H et al., “Efficacy of Botulinum-A Toxin in children with Detrusor Hyperreflexia Due to Myelomeningocele: Preliminary Results” Urology (2002) 59 (3); Schurch, B et al., “Botulinum-A Toxin for Treating Detrusor Hyperreflexia in Spinal Cord Injured Patients: A New Alternative to Anticholinergic Drugs? Preliminary Results” Journal of Urology (2002) 164, 2000; Ersay A et al., “Intravesical Oxybutynin Affects Bladder Permeability” International Urology and Nephrology (2001) 32, 359-361; Di Stasi, S et al., “Intravesical Oxybutynin: Mode of Action Assessed by Passive Diffusion and Electromotive Administration with Pharmacokinetics of Oxybutynin and N-Desethyl Oxybutynin” The Journal of Urology (2001) 166, 2232-2236

### SUMMARY OF THE INVENTION

Methods and compositions for treating urologic disorders are provided. In the subject methods, an effective amount of a neurotoxin is intravesically delivered into the bladder lumen of a patient in need thereof. Also provided are compositions, e.g., pharmaceutical preparations and kits, for practicing the subject methods.

It is an object of the present invention to provide a method of treating urologic disorders by exposing the lumen of the bladder and urinary tract to neurotoxin.

Another object of the present invention is to provide a method of the above character in which the neurotoxin is botulinum toxin.

Another object of the present invention is to provide a method of the above character in which a urethral catheter is placed to facilitate delivery of the neurotoxin into the lumen of the bladder and urinary tract.

Additional objects and features of the invention will appear from the following description from which the preferred embodiments are set forth in detail.

### DESCRIPTION OF THE SPECIFIC EMBODIMENTS

Methods and compositions for treating urological disorders are provided. In the subject methods, an effective amount of a neurotoxin is intravesically delivered into the bladder lumen of a patient in need thereof. Also provided are compositions, e.g., pharmaceutical preparations and kits, for practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Although any methods, devices and materials similar or equivalent to those described herein can be used in the practice or testing of the invention, the preferred methods, devices and materials are now described.

All publications mentioned herein are incorporated herein by reference for the purpose of describing and disclosing the elements that are described in the publications which might be used in connection with the presently describedinvention.

As summarized above, the subject invention provides methods and compositions for use in the treatment of urological disorders. In further describing the subject invention, the methods are described first in greater detail, followed by a description of the compositions, e.g., pharmaceutical preparations and kits, that find use in practicing the subject methods.

### Methods

As summarized above, the subject invention provides methods of treating urologic disorders, e.g., the intravesical administration of neurotoxin including botulinum toxin to treat urologic disorders in both males and females, as described in greater detail below. “Urologic disorders” includes, but is not limited to, detrusor instability, detrusor hyperreflexia, overactive bladder, neurogenic bladder, and urinary incontinence (of all types including but not limited to urge incontinence, stress incontinence, overflow incontinence, and mixed incontinence). “Urologic disorders” also includes, but is not limited to: irritable, spastic, unstable, hypertonic, uninhibited, dyssynergic, and systolic bladder. In addition, any urologic disorder that may benefit from a reduction in the frequency and amplitude of bladder contractions may be treated by the methods described.

A feature of the subject invention is that an effective amount of a neurotoxin is intravesically administered to the patient in need thereof. By “intravesically administered” or “intravesical administration” is meant instillation of pharmaceutical preparation or medication into the lumen of the bladder by any suitable means. Intravesical administration excludes, however, injection of medication into the wall of the bladder. Administration of an effective amount of a neurotoxin by intravesical administration reduces muscular bladder contractions, thereby reducing or eliminating symptoms and complications of the above mentioned urologic disease states.

The neurotoxin employed in the subject methods can be any which inhibits acetylcholine release from nerve endings. Other neurotoxins that find use include those that inhibit release of other neurotransmitters including, but not limited to: oxide, glycine, GABA, serotonin, dopamine, epinephrine, and norepinephrine from nerve terminals. Representative neurotoxins include botulinum toxin, tetanus toxin, tetrodotoxin, bungotoxin, terodotoxin, conotoxin and derivatives thereof, etc.

In certain embodiments, the neurotoxin that is employed is a botulinum toxin. Any or all of the botulinum toxins (A,B,C,D,E,F,G etc) may be used either alone or in combination. Suitable botulinum toxin is commercially available from Allergan (Irvine, Calif.) under the trade name Botox, and from Elan (Dublin, Ireland) under the trade name MyoBloc.

The dosage of neurotoxin agent that is intravesically administered to the patient during practice of the subject methods is one that is effective to achieve the desired treatment outcome. The amount of any particular agent will vary depending on the nature of the particular agent, and can be readily determined empirically by those of skill in the art. In the case of botulinum toxin, the typical dose administered to the patient may be any dose less than a toxic dose (for example less than 3000 units for a 70 kg man), preferably between 1 and 1,500 units and more preferably between 50 and 500 units per patient per treatment, although smaller or larger doses may be administered in appropriate circumstances. The doses can be given as a single dose, or as divided doses over a span of hours, days, or weeks.

The neurotoxin agent is intravesically administered to the patient in need thereof in a suitable pharmaceutically acceptable vehicle. In other words, the active neurotoxin agent is intravesically administered to the patient in a pharmaceutical preparation that includes the agent in a pharmaceutically acceptable vehicle. The active agent can be presented as a sterile pyrogen-free aqueous solution or dispersion or as a sterile powder for reconstitution into a sterile solution or dispersion. Where desired, tonicity adjusting agents such as sodium chloride, glycerol and various sugars can be added. Stabilizers such as human serum albumin can also be includedas can agents to alter or stabilize pH. Substances that promote adhesion of the neurotoxin to the mucosa may also be added. The formulation may be preserved by means of a suitable pharmaceutically acceptable preservative such as a paraben, although preferably it is unpreserved. It is preferred that the neurotoxin is formulated in unit dosage form.

The neurotoxin may be administered with one or more compounds that will increase or decrease the permeability of the neurotoxin (i.e., a neurotoxin permeability modulating agent) into the mucosa, submucosa, and/or muscle layer within the bladder wall. For example, an amine or other compound may be instilled intravesically either before, during, or after instillation of the neurotoxin in order to improve permeability of the neurotoxin into the bladder mucosa and nearby tissues. These one or more permeability modulatory agents may be administered to the host before, simtultaneously with, or after the neurotoxin is administered to the host.

The neurotoxin may also be administered with another agent designed to treat urologic conditions (i.e., neurotoxin or non-neurotoxin urologic condition active agents), including but not limited to oxybutynin and tolterodine. These agents may provide immediate, short-term relief (for overactive bladder for example) while the neurotoxin provides more long-term relief of symptoms. Alternatively, the agent may be a second neurotoxin that works through a similar or different mechanism to provide short or long term relief of symptoms.

The neurotoxin can be formulated in any pharmaceutically acceptable formulation and in any pharmaceutically acceptable form. Such forms and formulations include liquids, powders, creams, emulsions, pills, troches, suspensions, solutions, and the like. The neurotoxin can also be used in any pharmaceutically form supplied by any manufacturer.

One representative method of intravesically administering the neurotoxin involves the use of a urinary catheter that extends through the urethra into the bladder. The catheter may be a “straight catheter” with a single lumen or alternatively might be a multi-lumen catheter that in some cases uses a balloon or other mechanism to fix the catheter within the bladder. Standard sizes for such a catheter are 10-16 French although larger or smaller sizes might be used depending on the sex of the patient and his or her anatomy. Once the catheter is in place, between 1 and 1000 ml of solution/dispersion containing neurotoxin and more preferably in the range of 10-50 ml of solution/dispersion containing neurotoxin can be instilled through the catheter into the bladder. The volume of the solution/dispersion and concentration of the neurotoxin may depend upon the size of the patient, thickness of the bladder wall and muscle, comorbidities, and other factors.

Because the patient will likely be instructed to empty his or her bladder prior to the procedure, the bladder will likely not be full or markedly distended. As such, it is expected that instillation of 1-100 ml of solution/dispersion, and more preferably 10-50 ml of solution/dispersion, may be sufficient to coat the inside of the bladder. The patient may be asked to position himself or herself in various positions to ensure that the solution/dispersion containing the neurotoxin makes contact with all surfaces on the inside of the bladder. In some cases, the catheter will be removed and the patient will be asked to void his or her bladder to expel the neurotoxin and any accumulated urine. In this case, the distal portions of the urinary tract including the bladder neck, sphincter and urethra may come in contact with and derive therapeutic benefit from exposure to the neurotoxin.

Another representative means of intravesically administering the neurotoxin involves the placement of a suprapubic needle or catheter through the abdominal wall directly into the bladder. In this case, the requisite volume of fluid containing the neurotoxin is introduced into the bladder, either using direct vision, or endoscopic or fluoroscopic guidance.

In some cases, the urethral or suprapubic catheter/needle may have an inflatable component that can be inflated within the bladder. Inflating the balloon or other inflatable device is intended to take up volume within the bladder, thereby requiring less solution/dispersion containing the neurotoxin to fill the lumen of the bladder.

Another representative means of intravesically administering the neurotoxin is through the use of a cystoscope, wherein the cystoscope facilitates viewing of intravesical delivery of the medication. In this case, the solution/dispersion containing neurotoxin can be introduced into the bladder lumen through the working channel of the cystoscope or through a catheter or other tubular structure passed within or alongside the cystoscope.

Alternatively, the neurotoxin may be deposited within a reservoir or other device capable of sustained release of the neurotoxin over hours, days, weeks or longer time frame. This reservoir could be left within the bladder for hours, days, weeks, or years. The reservoir may be in the form of a mechanical device, polymeric compound, or non-polymeric compound or the like that would allow release of toxin over an extended time frame. In some cases, the device or compound may also function as an adhesive, attached to a portion of the bladder wall or urinary tract.

In other cases, the toxin may be mixed with a compound capable of adhering to the mucosa within the urinary tract. This would allow the toxin to be retained within the bladder for a longer period than in the absence of such a compund. A representative compound capable of adhering to the the urinary tract/bladder mucosa is hydroxypropylcellulose.

In some cases, the neurotoxin may come partially or completely pre-loaded in a delivery system. This format allows the user to quickly and safely administer the neurotoxin. In some cases, this format might mean the solution/dispersion containing neurotoxin may come pre-loaded in a syringe, requiring the user to merely connect the syringe to the delivery system/device. In other cases, the delivery system may be a completely closed system. By completely closed is meant a device that makes it unlikely or impossible for the user to come into contact with the neurotoxin. This makes it less likely that the patient or healthcare provider will accidentally come into contact with a potentially harmful compound.

The solution/dispersion containing the neurotoxin may be kept in the bladder and/or urinary tract anywhere from 5 seconds to many hours, although most likely, it will be either voided by the patient or removed by catheter or other means within 30 minutes to 3 hours. The voided fluid must be discarded carefully because it contains neurotoxin that might be harmful to the patient and others.

In some cases, electromotive drug administration may be used to increase the amount of neurotoxin that is absorbed into the bladder/urinary tract's tissues. This may involve the use of a catheter with a electrode near its tip. Alternatively, the solution/dispersion may be heated or cooled to increase absorption of the neurotoxin into the bladder, etc.

A physician, nurse, nursing assistant, physician's assistant, nurse practitioner, medical student or other healthcare provider, will most likely carry out the procedure. Depending on the circumstance, it might be possible for the patient or a non-healthcare provider to perform the procedure.

The subject methods find use in the treatment of a variety of different urologic disorders, as summarized above. By “treatment” is meant that at least an amelioration of the symptoms associated with the urologic disorder afflicting the host is achieved, where amelioration is used in a broad sense to refer to at least a reduction in the magnitude of a parameter, e.g. symptom, associated with the urologic disorder being treated. As such, treatment also includes situations where the urologic disorder, or at least symptoms associated therewith, are completely inhibited, e.g. prevented from happening, or stopped, e.g. terminated, such that the host no longer suffers from the disorder, or at least the symptoms that characterize the condition.

Following the procedure, there is little or no unwanted systemic or local side effects. Furthermore, the treated patients show an improvement in bladder function and symptomatology both subjectively and when measured objectively.

Representative animals with which these devices and methods find use include, but are not limited to: canines, felines, bovines, ovines, etc. and primates, particularly humans.

### Pharmaceutical Preparations

Also provided by the subject invention are pharmaceutical preparations, as described above. The pharmaceutical preparations include an effective amount of a neurotoxin agent in a suitable intravesical delivery vehicle, where representative embodiments of each of these components are described above in greater detail.

### Kits

Also provided are kits for use in practicing the subject methods. The subject kits at least include a pharmaceutical preparation, as described above. The pharmaceutical preparation is typically present in the kit in a suitable container, e.g., a bottle, pouch, etc. The kits may also include in intravesical delivery device for use in intravesically administering the provided pharmaceutical preparation to a patient.

In addition to the above components, the subject kits further include instructions for using the components in the treatment of urological disorders, as described above. The instructions are generally recorded on a suitable recording medium. For example, the instructions may be printed on a substrate, such as paper or plastic, etc. As such, the instructions may be present in the kits as a package insert, in the labeling of the container or the kit or components thereof (i.e. associated with the packaging or subpackaging) etc. In other embodiments, the instructions are present as an electronic storage data file present on a suitable computer readable storage medium, e.g. CD-ROM, diskette, etc. The instructions may take any form, including complete instructions for how to use the device or as a website address in which instructions may be accessed.

Any means by which a neurotoxin is intravesically introduced into the lumen of the bladder or urinary tract to treat a urologic disorder or disease falls within the spirit and scope of this application.

The foregoing description of the invention is exemplary for purposes of illustration and explanation. It will be apparent to those skilled in the art that changes and modifications are possible without departing from the spirit and scope of the invention. It is intended that the following claims be interpreted to embrace all such changes and modifications.

The invention will now be illustrated by reference to the following nonlimiting examples.

### EXPERIMENTAL

### EXAMPLE 1

Intravesical use of Botulinum toxin Types A to G in the Treatment of urinary incontinence.

A female patient, age 50, suffering urinary incontinence, is treated intravesically with 250 units of Botulinum toxin type A in an aqueous medium through a urethral catheter. The catheter is removed and the patient voids the toxin after 1.5 hours. After a period of 1-3 days, the symptoms of urinary incontinence, i.e., urgency, frequency, leaking etc. are markedly reduced.

### EXAMPLE 1(a)

The method of Example 1 is repeated, except that a patient suffering from urinary incontinence is treated intravesically with 50-500 units of Botulinum toxin type B. A similar result is obtained.

### EXAMPLE 1(b)

The method of Example 1 is repeated, except that a patient suffering from urinary incontinence is treated intravesically with 50-500 units of Botulinum toxin type C. A similar result is obtained.

### EXAMPLE 1(c)

The method of Example 1 is repeated, except that a patient suffering from urinary incontinence is treated intravesically with 50-500 units of Botulinum toxin type D. A similar result is obtained.

### EXAMPLE 1(d)

The method of Example 1 is repeated, except that a patient suffering from urinary incontinence is treated intravesically with 50-500 units of Botulinum toxin type E. A similar result is obtained.

### EXAMPLE 1(e)

The method of Example 1 is repeated, except that a patient suffering from urinary incontinence is treated intravesically with 50-500 units of Botulinum toxin type F. A similar result is obtained.

### EXAMPLE 1(f)

The method of Example 1 is repeated, except that a patient suffering from urinary incontinence is treated intravesically with 50-500 units of Botulinum toxin type G. A similar result is obtained.

It is evident from the above results and discussion that the subject invention provides for a number of advantages over currently employed methods of pharmacologically treating urological disorders. Such advantages include the minimally invasive aspects of intravesical delivery and the infrequent administration schedule provided by the use of the neurotoxin agent, where these advantages will result in a significantly improved treatment protocol, particularly from the vantage of the patient. Accordingly, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of treating a host suffering from a urologic disorder, said method comprising:
intravesically administering to said host an effective amount of a neurotoxin to treat said host for said urologic disorder.

2. The method according to claim 1, wherein said host is a human.

3. The method according to claim 1, wherein said urologic disorder is a bladder disorder.

4. The method according to claim 3, wherein said bladder disorder is urinary incontinence.

5. The method according to claim 1, wherein said neurotoxin is a botulinum toxin.

6. The method according to claim 1, wherein said method further comprises intravesically administering at least one neurotoxin permeability modulating agent to said host.

7. The method according to claim 6, wherein said at least one neurotoxin permeability modulating agent is administered to said host prior to said neurotoxin.

8. The method according to claim 6, wherein said at least one neurotoxin permeability modulating agent is administered to said host after said neurotoxin.

9. The method according to claim 6, wherein said at least one neurotoxin permeability modulating agent is administered to said host simultaneously with said neurotoxin.

10. The method according to claim 1, wherein said method further comprises administering to said host at least one additional agent to treat said urologic condition.

11. The method according to claim 1, wherein said neurotoxin is administered to said host with a catheter.

12. The method according to claim 11, wherein said catheter comprises an inflateable component and said method comprises inflating said inflateable component prior to, during, or after administration of said neurotoxin.

13. The method according to claim 1, wherein said neurotoxin is administered in a reservoir.

14. The method according to claim 13, wherein said reservoir is a mechanical device.

15. The method according to claim 13, wherein said reservoir is a polymeric composition.

16. The method according to claim 1, wherein said administering includes electromotive drug administration.

17. A pharmaceutical preparation for use in treating a urologic disorder, said preparation comprising: an effective amount of a neurotoxin in an intravesical delivery vehicle.

18. The pharmaceutical preparation according to claim 17, wherein said neurotoxin is a botulinum toxin.

19. An intravesical delivery device loaded with a pharmaceutical preparation according to claim 17.

20. The device according to claim 19, wherein said device is a catheter.

21. A kit for use in treating a urological disorder, said kit comprising:
a pharmaceutical preparation according to claim 17; and
instructions for using said preparation in a method according to claim 1.

22. The kit according to claim 21, wherein said kit further comprises an intravesical delivery device for intravesically delivering said pharmaceutical preparation to a host.

23. The kit according to claim 22, wherein said device is a catheter.
